(19) United States
(12) Patent Application Publication
(10) Pub. No.: US 2017/0172928 A1
(43) Pub. Date: Jun. 22, 2017

(54) PHARMACEUTICAL SYSTEM FOR ORAL DELIVERY OF SENSITIVE THERAPEUTIC SUBSTANCES

(71) Applicant: UNIVERSIDAD NACIONAL AUTONOMA DE MEXICO, Distrito Federal (MX)

(72) Inventors: David QUINTANAR GUERRERO, Cuautitlan Izcalli Estado de Mexico (MX); Laura Elizabeth HERNANDEZ RIZO, Distrito Federal (MX)

(21) Appl. No.: 15/129,714

(22) PCT Filed: Mar. 25, 2015
(86) PCT No.: PCT/MX2015/000052

§ 371 Date: Sep. 27, 2016

(30) Foreign Application Priority Data
Apr. 1, 2014 (MX) MX/A/2014/003939

(51) Int. Cl. A61K 9/28; B05D 5/00

(52) U.S. Cl. CPC A61K 9/284; A61K 9/2853; A61K 9/2866; A61K 9/2826; B05D 5/00; A61K 2121/00

(57) ABSTRACT

The present invention specifically refers to a pharmaceutical system for preparation of solid pharmaceutical forms for administering therapeutic substances improving their bioavailability; said system is particularly suitable for administering sensitive active principles by their proteinaceous nature or by being nucleic acids since polymeric excipients with good matrix and bioadhesion properties are used which together with the impermeability conferred by the applied coating make the prepared system a delivery form with excellent performance.

### FIELD OF INVENTION

The present invention relates to the field of controlled release systems for delivery of active substances and to solid pharmaceutical form coatings.

### BACKGROUND OF INVENTION

Several drugs show a low oral bioavailability, therefore they are not suitable candidates at first for this route of administration. This is due to instability in stomach strongly acid medium, to the effect caused by intestinal peptidases and especially to a poor permeability as consequence of molecular size, electric charge and high polarity (Norris, et. al.).

Physiological factors such as gastrointestinal transit time, dilution and interactions with food components reduce the available dose fraction for peptide and protein absorption. Finally, the “first step” effect and “flow pumps” are responsible of the limited oral bioavailability shown by this type of active principles.

A number of strategies have been developed in order to increase oral bioavailability of peptides and proteins by controlling some of above mentioned factors. Structural changes to modify the polarity or to reduce the “first step” effect without compromising their biological activity are highlighted among them.

With the same goal this has resorted to the association with bioadhesive substances which sensitively delay intestinal transit and increase contact time with mucous membrane surface. Bioadhesive polymers increase macromolecule absorption by bioadhesion and modulate the permeability by acting into intercellular spaces. Oral delivery bioadhesive systems are targeted to fix into stomach or intestinal mucous membrane and continuously supply a drug dose to be absorbed in small intestine (Brayden, et. al.).

There have been several attempts up to date to prepare pharmaceutical systems allowing administration of degradable active substances, such as those described below.

International publication WO 95/06077 (Acemoglu, et. al.) defines a new polyethylene carbonate biodegradable polymer to shape pharmaceutical composition matrices to release active component in controlled manner in the form of microparticles or implants and they mention that it may be a peptide or a protein; however these matrices are for parenteral administration.

International publication WO 2004/108067 A2 (Dharmadhikari & Zala) describes a programmed drug release system. The system consists of a core containing at least one excipient which is swollen when contacting water, a coating preventing passage of core components but that may be water permeable or impermeable and a duct allowing water entry; cellulose derivatives, polyvinyl pyrrolidone polymers, crospovidone, natural and artificial gums as well as their copolymers are used; said system being useful for purposes of delayed, controlled or targeted release but more particularly to drug release targeted to certain gastrointestinal tract zones where the therapeutic substance should act. It is worth to remark that the system corresponding to WO 2004/108067 A2 publication, is limited by duct dimensions, said duct being in the center of the pharmaceutical form allowing medium entry to perform dissolution and drug release, it may be easily obstructed by polymer swelling and is technologically difficult to perform, particularly when thinking in an industrial process. Furthermore, the duct located at core center is in charge of making that the cover is impermeable or not.

U.S. Pat. No. 4,892,739 (Shah & Koparkar) also proposes a continuous release oral delivery system where from 50 to 90% weight of drug is released into gastrointestinal tract contained at a rate from 5 to 12% weight per hour, said system comprising a solid center containing an osmotically active composition and an active agent being a beta blocker which may be further in the presence of another excipient, then comprising a cover being a water-soluble and permeable polymeric binder at a 0.3 to 10% ratio of total center weight, and an impermeable membrane to gastrointestinal fluids outside surrounding the center and the permeable cover and finally possessing at least a duct in this impermeable membrane so that the active agent has communication with outer environment. This patent design in addition to being limited to carry a beta blocker as active agent, differs with the present invention in requiring that the osmotically active agent in contact with gastrointestinal fluids may exhibit a gradient to apply pressure against the outer cover thus promoting the passage of active agent from system center to outer side, it is an osmotic system which functioning fully depends on the osmotic agent included in the pharmaceutical system core and on the pressure which may be generated inside the system.

U.S. Pat. No. 5,439,688 (Martigny & Villeneuve) describes a preparation process for a pharmaceutical composition in form of microparticles or implants based on polyester-type biodegradable copolymers, said preparations having use in controlled release formulations. Design is different from that of present invention and it is worth to mention that in spite of the properties of obtained compositions, the main disadvantage of this process resides in that such technique involves the use of organic solvents and in many cases there are traces of them in the preparations, making difficult their use for administering therapeutic substances.

U.S. Pat. No. 6,468,959 B1 (Wunderlich, et. al.) refers to a pharmaceutical form for administering proteinaceous type substances, comprising matrices based on gelatin or gelatin derivatives due to their capacity of adhering into the intestinal mucous membrane. However, the existence of charges is decisive for this kind of systems so that the system functions under the expected mode.

European publication EP 0274734 A1 (Colombo, et. al.) describes the use of multilayer pills for active substance repeated release. This kind of systems is widely useful in controlled release formulations but limited to inclusion of non-degradable drugs; where in addition to not having intimate and retentive contact between the dosage form and mucous membrane the drug is not protected with an impermeable barrier therefore its use may not be extended to sensitive molecules. Furthermore, this system only functions by disintegration of multiple layers where the drug is included.

U.S. Pat. No. 5,430,021 (Rudnic, et. al.) suggests a pharmaceutical composition where active substance is incorporated into finely divided hydrophobic particles which are coated and incorporated into a dosage form for oral delivery. This invention has a slow release limitation which may be generated from those systems.

The work disclosed by Benkorah & McMullen describes the use of a biconcave core coated with polycaprolactone by a fusion-injection method which disadvantage is the generated membrane rigidity; being a pharmaceutical form based on conventional excipients having an impermeable but quite rigid coating where drug release is from a central duct in the pharmaceutical form.

The present invention refers to a pharmaceutical system for oral delivery of sensitive therapeutic substances, that is, actives which are susceptible to degradation due to characteristic conditions of gastrointestinal tract such as, gastrointestinal transit, enzymatic activity, pH gradient, etc., through the implementation of polymer technology and application of an organic film coating, used materials provide good impermeability, bioadhesion and swelling properties to the system which are favorable for active substance controlled release. Moreover, a direct contact between the pharmaceutical system and mucous membrane is promoted with this system, thus contributing to provide any unidirectional type release.

In the light of above, it is intended to suppress drawbacks shown by oral delivery pharmaceutical systems of prior art, by developing a controlled release system for administering active substances and solid pharmaceutical form coatings.

### OBJECTS OF THE INVENTION

Taking into account the implications of the state of the art, it is an object of the present invention to provide a controlled and unidirectional release system characterized by its physical design thanks to which, the solid pharmaceutical form manufactured under this design shows both bioadhesion and impermeability simultaneous potential properties to generate an intimate contact between the pharmaceutical system and the application site.

Moreover, it is another object of the present invention to provide a controlled and unidirectional release system without being in contact with biological fluids by the non-adhered impermeable part or which does not have any intimate contact with absorption mucous membrane.

A further object of present invention is to provide a controlled and unidirectional release system preventing active component degradation before being assimilated.

### BRIEF DESCRIPTION OF THE DRAWINGS

Novel aspects which are deemed characteristic of present invention are specifically established in the attached claims. However, some embodiments, features and some objects and advantages thereof will be better understood in the detailed description when read in relation to the attached figures wherein:

FIG. 1 shows a schematic representation of release system functioning of the invention where its parts are indicated: the impermeable outer membrane and matrix system.

FIG. 2 shows a schematic representation of a spraying device in a rotary cylinder. [A] Air inlet (DeVILBISS, U.S.A.), [B] stirrer (Amphenols Control Div. U.S.A.), [C] dispersion, [D] magnetic stirrer (Barnstead Int. U.S.A.), [E] peristaltic pump (Masterflex, Mexico), [F] spray gun (Walter Pilot, México), [G] rotary drum (15 cm high, 30 cm diameter), [H] drying system (Milwuakke, Mexico), and [I] infrarred thermometer (OAKTON®, Temptestr® IR, Mexico). Image taken from the work Comparison of Pharmaceutical films prepared from aqueous polymeric dispersion using the cast method and the spraying technique. (Mendoza, et. al.)

FIG. 3 shows a number of photographs of polymer swelling during active principle release from 0 to 24 hours.

FIG. 4 shows a graph of the sensitive drug release profile in mg from the release system in function of time with coating and without coating.

### DETAILED DESCRIPTION OF THE INVENTION

The present invention has the purpose of offering a non-conventional, unidirectional pharmaceutical system whether for oral or topical administration of drugs or therapeutic substances, particularly those which are sensitive or susceptible to outer environment degradation (e.g., peptides, proteins, genes, etc.). That is, drugs which are susceptible to degradation by effect of gastrointestinal tract characteristic conditions, mainly: gastrointestinal transit itself, enzymatic activity, and pH gradient.

Environments where invention may be applicable are for instance: generally the gastrointestinal tract lumen, including oral environment when administration is sublingual, stomach environment, duodenum, jejune, ileum lumen, and anal-rectal mucous membrane and also may be applied by administration to vaginal passage.

The invention is designed to allow diffusion of drugs, active principles or therapeutic agents towards patient's systemic blood stream through their transport by mucous membrane cell membranes being passively or actively diffused; this promotes a higher bioavailability of active principle, being comprised those active principles which are not of easy absorption by mucous membrane and which further may be supported by a carrier molecule allowing transportation depending on the type of active principle. The delivery system of the invention allows a single and efficient administration leading to a reduction in number of intakes/day by providing controlled release and bioadhesion to mucous membranes. Bioadhesion is understood as natural or synthetic polymer binding to a biological substrate. When the substrate is a mucous membrane layer as in gastrointestinal tract, the term mucoadhesion is frequently used. Mucoadhesion has been widely promoted as a form of achieving drug release at specific site through inclusion of mucoadhesive hydrophobic polymers together with an active principle within the pharmaceutical form. The main reason for the formulation to be located in a biological surface is to perform a located drug release.

The present invention particularly refers to a novel pharmaceutical system for administering sensitive therapeutic substances, having good matrix and bioadhesion properties which together with impermeability conferred by the applied film (organic) coating make the system a promising tool for oral delivery of sensitive active substances such as peptides, proteins, nucleic acids and the like, as well as any active of variable chemical nature requiring protection and/or bioadhesion.

Advantages of the proposed system are that due to the type of used excipients and their packaging, an intimate contact is promoted between the pharmaceutical system and the mucous membrane in question, thus allowing an unidirectional type controlled release to result allowing an increase in residence time of the action system and therefore an unidirectional type controlled release of the active substance. Unidirectional release is being understood as that made directly into the absorption site (e. g. mucous membranes). Drug release is made in different directions in a conventional system since all the pharmaceutical form is in contact with the medium. As to the developed system it is intended to protect the pill convex face to be the flat face thereof which contacts the mucous membrane and is bioadhered thus active release is only made into the mucous membrane, this being the meaning of the term “unidirectional”. Additionally, the applied film coating confers a suitable impermeability to the system for protection of sensitive or degradable active substances.

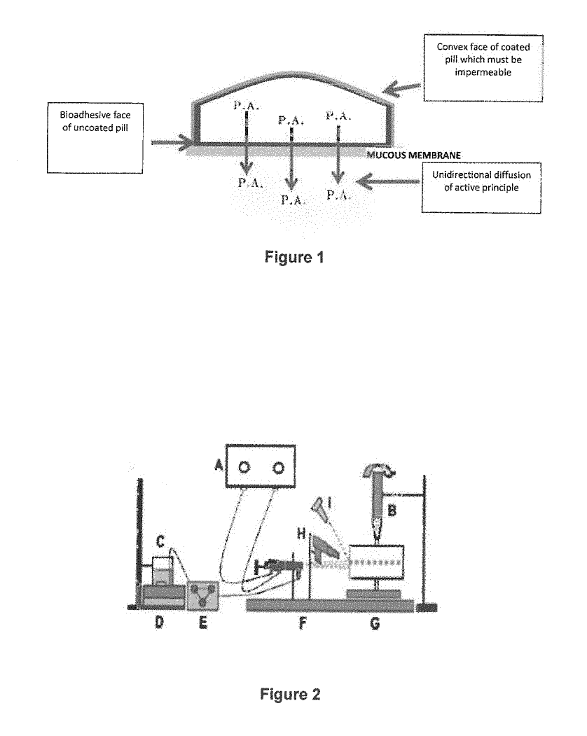

FIG. 1 is a schematic representation of the pharmaceutical system described in present invention. As it may be noticed, the system is formed by a core with matrix and bioadhesive properties where the therapeutic substance is to be included and which is protected by one of its faces (convex face) by an impermeable membrane or coating formed by a plasticized lipophilic polymer (e. g. polycaprolactone with polyethylene glycol). As illustrated in FIG. 1, the flat section of the system remains without impermeable coating and is exposed whereby when adhered to the mucous membrane becomes into a flattened form and upon contacting the mucous membrane a release of the therapeutic substance is only caused in that section.

In the core of the invention's system, an acrylic acid crosslinked polymer (commercially known as Carbopol 934®) which is a hydrophylic polymer is used, providing the desired matrix properties for this system; while the dihydrated calcium dibasic phosphate or CaHPO₄.2H₂O, commercially known as (Ditab®) which is also used in the core due to a low solubility, allows a gradual swelling of said hydrophylic polymer, the ratio between them (Carbopol 934® and Ditab®) is a 70:30 ratio.

Moreover, the applied coating comprising poly-ε-caprolactone (20%), and polyethylene glycol (20%), provides an impermeable film to the pharmaceutical system, so that while not being coated on its flat face, the release of active principle is unidirectionally and directly controlled to the mucous membrane as previously described. One of the preferred embodiments of the invention refers to a coating formulation with the following components:
a) poly-ε-caprolactone (20%) as hydrophobic polymer,
b) polyethylene glycol and diethyl phtalate as plasticizer (20% with respect to polymer),
c) ethyl acetate as solvent.

The area which is free for contact with the mucous membrane is 1.9007 cm², but this may vary to any dimension according to the type of die being used to prepare the pill; a reasonable range is from 0.5 mm to centimeters (e.g., 3 cm).

FIG. 2 shows an equipment type scheme used for coating the pharmaceutical system subject of present invention.

According to the present invention, the pharmaceutical system consists of:
a) Core or matrix based on excipients with matrix and bioadhesive properties where the therapeutic substance which may be one or more active principles will be included.
b) An impermeable membrane or coating.

A set of die with matrix was designed and manufactured for obtaining the proposed system, allowing obtaining a core with a concave shape upper face and a flat inner face, as illustrated in FIG. 1; the objective of obtaining a core with previously mentioned features is that the pill concave face is coated with specific polymers and therefore impermeable, and that the flat face without coating establishes a direct contact with mucous membrane allowing an unidirectional controlled release of the therapeutic substance at action site.

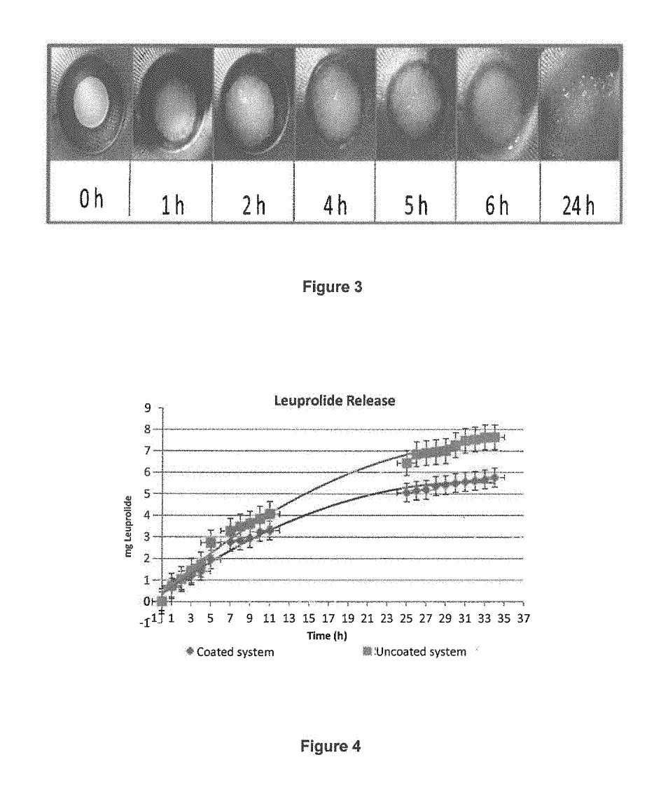

FIG. 3 illustrates photographic images of swelling test where noticed the progressive polymer swelling which leads to a matrix behavior of the system.

The graph in FIG. 4 shows the trend followed by active principle release which together with the executed mathematical model indicates that it has a matricial behavior. Models: Higuchi: k=0.1444, R=0.9963; Peppas: n=0.6187, R=0.9920.

Objective of present invention is to provide a controlled release and unidirectional system being characterized by its physical design thanks to which the solid pharmaceutical form which is manufactured based on this design shows simultaneous potential properties of both bioadhesion and impermeability, in order to generate an intimate contact between the pharmaceutical system and the application site (e.g., gastrointestinal tract, vaginal, oral mucous membrane) and therefore an unidirectional release without being in contact with biological fluids by the non-adhered impermeable part or which does not have intimate contact with absorption mucous membrane, the system of the invention prevents active component degradation before being assimilated.

It is worth to remark that the system disclosed in document WO 2004/108067 A2, corresponding to the closest state of the art, is very different from the system of present invention, where in addition to the features already described in the background, it is a system where an adhesion process with the intestinal mucous membrane is not involved therefore the drug release effect is different, the system of the present invention has a larger surface where release is occurring, it is a smooth (flat) area responsible of bioadhesion, with this design and unlike the system described in WO 2004/108067 A2, it is not susceptible of being plugged. Likewise, with the system of the present invention, excipient ratio allows a suitable swelling with good bioadhesive properties and manufacturing and scale-up feasibility has been apparent.

It is important to remark that U.S. Pat. No. 4,892,739 cited in background, describes a system where an adhesion process from the part where the duct is directly with the intestinal mucous membrane is not involved, therefore the effect of drug release is different from that disclosed in our invention; our system complies with a matricial behavior resulting from powder mixing, equivalent to the conventional pill: there are ingredients of two kinds: a hydrophilic polymer (Carbopol) which will swell and an insoluble ingredient (Ditab®) allowing mixture compression; swelling is smooth and generates an active principle controlled release.

It is also important to remark that system proposed in U.S. Pat. No. 6,468,959 B1 cited in background as part of the closest state of the art, differs from our system as to performance. In order for this system to function properly it is necessary that the active is complexed and with an opposite charge to that from gelatin which is part of it. On the other hand, the system we are proposing includes the free active principle in the core, and release is carried out by a diffusion mechanism due to the matrix properties provided by Carbopol® and Ditab® (or similar compounds thereof). On the other hand, Carbopol bioadhesion has been widely reported by an interpenetration between polymer molecules and those present in mucous membrane mucus therefore, neither pH conditions nor the presence of another molecule are required.

As mentioned, the proposed system in European publication EP 0274734 A1 of the state of the art cited in the background is limited to the inclusion of non-degradable drugs as it is not protected by any kind of barrier; moreover, the system functions as already mentioned, by disintegration of the layers and not because of diffusion and bioadhesion phenomena as in the case of the system proposed in present invention.

In the system described by Benkorah & McMullen cited in polycaprolactone which coating is impermeable but very rigid and where the drug release results from a central duct in the pharmaceutical form, any bioadhesive effect is not mentioned and much less an unidirectional release thus the drug in fact is not fully protected unlike the system of the present invention, protecting the drug under gastrointestinal tract unfavorable conditions and also contributing to unidirectional type active principle release.

An embodiment of the invention, in addition to providing an active substance controlled release system, relates to the means and steps for manufacturing solid pharmaceutical forms for oral delivery or topical use based on this unidirectional and controlled release pharmaceutical system.

Embodiments of the invention are also those which further to described design, they also involve inclusion of polymeric materials which may be used to form an impermeable coating, these are pharmaceutically acceptable polymers for topical administration where bioadhesion may be present, mainly designed for oral delivery; and which may be for instance: Poly-ε-caprolactone, ethyl cellulose, polyacrylates and their copolymers, shellac, cellulose, polylactic acid and polyglycolic acid and their copolymers, among many other water insoluble polymers, including mixtures thereof.

It is within the embodiments the use of poly ε-caprolactone bioadherent polymer dissolved in ethyl acetate and the addition of diethylphtalate plasticizer.

Embodiments of the invention are in addition to described design, the polymeric materials which may be used to form the core or matrix which have bioadherent properties certainly of pharmaceutical acceptance such as: Carbopols, polyvinyl polyalcohol, polyvinylpyrrolidone, povidone, cellulose derivatives (e.g. methyl cellulose, methyl hydroxyethyl cellulose, carboxymethyl cellulose, hydroxypropyl methyl cellulose, etc.) chitosan, gelatin, gums (e.g. guar, xanthan, arabic, etc.), among many other polymers reported with adhesive properties.

Embodiments of the invention are, the inclusion of one or more active principles in the pharmaceutical form which are topically administered through mucous membranes, with several functions which may include: functions in digestive tract and metabolism, cardiovascular, dermatologic, blood functions, functions in genital-urinary system, systemic anti-infection agents, antineoplastic agents and immunomodulators, with functions in muscle-skeletal system, respiratory system, sensory organs, antiparasitic agents, and the like.

In an embodiment of the invention, the design of the solid pharmaceutical form in pill form of sensitive drugs allowing oral delivery provides in these pharmaceutical forms for 90% of the system being coated and the remaining 10% being available for bioadhesion when contacting the mucous membrane.

### Examples

The present invention will be better understood from the following examples, which are only presented with illustrative purposes to allow a full understanding of the preferred embodiments de the present invention, without excluding that there are other embodiments not illustrated which may be put into practice based on the previously realized detailed description.

Following examples are illustrative of the properties, capacities and scope of the invention, but are not limitative in its true scope. The following data and experimental results are described in order to provide the necessary elements to carry out the invention, though not being limitative of the scope thereof:

### Example 1. Core ‘Preparation

1. Excipient or a mixture of excipients is weighed in an analytical balance for direct compression.
2. Matrix filling with previously weighed powders.
3. Direct compression in a laboratory hydraulic press (Carver Press).

### Example 2: Core Coating

A device proposed by the Research and Postgraduate Laboratory in Pharmaceutical Technology (Mendoza, et. al.) illustrated in FIG. 2 may be used for core coating. The illustrated apparatus has the advantage of allowing an easy and quick modification of any critical variables involved in coating such as drying temperature, spraying flow and pressure while film coating operations are simulated. Functioning conditions were optimized but they may be modified depending on the intended purpose.

In this preparation example of the system of the invention, the distance between the spray gun and the rotary cylinder shall be about 15.5 cm, assuring coverage of at least 90% of rotary cylinder surface. The drying gun is arranged at 4 or 5 cm apart from the spraying gun to prevent any type of interference, as well as to keep substrate temperature.

Preparation of the coating solution then proceeds: poly ε-caprolactone polymer, solvent (ethyl acetate) and plasticizer (polyethylene glycol) are weighed in an analytical balance, using beakers as containers. Weighed components are mixed in a 50 ml metal glass and dissolved in double boiler with constant agitation during 5 minutes.

Coating application then proceeds: Equipment cleaning is checked. Coating equipment is assembled as illustrated in FIG. 2. Pills are arranged in the rotary cylinder with support of a dual face adhesive tape leaving a distance of 5 cm between each pill. Coating is applied at a pressure of 0.2 MPa and flow rate of 2 mL/min. Pills are dried during 24 hours and tests of rupture strength, friability, weight uniformity, swelling and the like are performed.

### Example 3: Preparation of a Controlled Release System

239.75 mg of Carbopol®, 102.75 mg of Ditab® and 7.5 mg of Leuprolide were weighed in an analytical balance and compressed in a laboratory hydraulic press (Carver Press) with 0.5 tons force per 30 seconds. Pills were reserved for further coating. A coating solution with poly-ε-caprolactone (20%) and polyethylene glycol (20%) was prepared, using ethyl acetate as solvent. Pills are arranged in the rotary cylinder with support of a dual face adhesive tape leaving a distance of 5 cm between each pill. Coating is applied at a pressure of 0.2 MPa and flow rate of 2 mL/min. Pills are dried during 24 hours and tests of rupture strength, friability, weight uniformity, swelling and the like are performed.

Even when certain embodiments of the invention have been illustrated and described, it should be remarked that numerous variations thereof are possible, but such variations would not represent being apart from the true scope of the invention. Therefore, the present invention shall not be considered restricted except for the provisions in the state of the art, as well as by the scope of the attached claims.

### REFERENCES

Acemoglu, M., Bantle, S., Bodmer, D., Cammisuli, S., Hiestand, P., Nimmerfall, F., y otros. (1995). Polymeric matrices and their uses in pharmaceutical compositions.
Benkorah, A., & McMullen, J.-N. (1994). Biconcave coated, centrally perforated tablets for oral controlled drug delivery. Journal of Controlled Release, 155-160.
Brayden, D., & O'Mahony, D. (1998). Novel oral drug delivery gateways for biotechnology products: polypeptides and vaccines. Pharmaceutical Science & Technology Today, 7 (1), 291-299.
Colombo, P., La Manna, A., & Conte, U. EP patent 0274734 A1.
Dharmadhikari, N., & Zala, Y. (2004). Programmed drug delivery system. International Publication WO 2004108067 A2.
Mendoza, L., Pinan, E., Nava, M., Ganem, A., Cordero, S., & Quintanar, D. (2009). Comparison of
Pharmaceutical films prepared from aqueous polymeric dispersion using the cast method and the spraying technique. Colloids and Surfaces A: Physicochem. Aspects, 337, 109-116.
Norris, D. A., Purin, N., & Sinko, P. J. (1998). The effect of physical barriers and properties on the oral absorption of particulates. Advanced Drug Delivery Reviews, 34 (2-3), 135-154.
Orsolini, P., & Heimgartner, F. (1995). U.S. Pat. No. 5,439,688 A.
Rodriguez, I., Cerezo, A., & Salem, I. (2000). Sistemas de liberacion bioadhesivos. Ars Pharmaceutica, 41 (1), 115-128.
Rudnic, E., McCarty, J., & Belenduik, G. (1995). U.S. Pat. No. 5,430,021 A.
Wunderlich, J.-C., Schick, U., Freidenreich, J., & Werry, J. (2002). U.S. Pat. No. 6,468,959 B1

1. A controlled release solid pharmaceutical form of at least one active principle comprising:
a) a core formulated with bioadhesive agents and at least one active principle,
b) a concave-shape core coating functioning as impermeable membrane to body fluids; and
c) a core flat portion being free of impermeable coating and allowing bioadhesion to absorption mucous membrane and unidirectional and controlled release.

2. The solid pharmaceutical form according to claim 1 wherein is orally and topically administrable through the intestinal mucous membrane.

3. The solid pharmaceutical form according to claim 1, wherein the active principle is sensitive to environment degradation which prevails in any section of the gastrointestinal tract.

4. The solid pharmaceutical form according to claim 1, wherein is formed as pill, tablet or lozenge.

5. The solid pharmaceutical form according to claim 1, wherein the active principle is of peptidic or proteinaceous nature, or is a nucleic acid.

6. The solid pharmaceutical form according to claim 1, wherein the impermeable coating is comprised by a mixture of polymers of poly-ε-caprolactone as hydrophobic polymer, polyethylene glycol and diethyl phtalate as plasticizer, and ethyl acetate as solvent.

7. The solid pharmaceutical form according to claim 1, wherein the core comprises Carbopols, polyvinyl polyalcohol, polyvinylpyrrolidone, povidone, cellulose derivatives, methyl cellulose, methylhydroxyethylcellulose, carboxymethyl cellulose, hydroxypropylmethyl cellulose, chitosan, gelatin, gums, guar, xanthan, or arabic.

8. The solid pharmaceutical form according to claim 7, wherein the core comprises an acrylic acid crosslinked polymer (Carbopol 934®), and dihydrated calcium dibasic phosphate or CaHPO₄.2H₂O, (Ditab®), in a 70:30 ratio.

9. A process for manufacturing a solid pharmaceutical form of claim 1, comprising the steps of:
a) preparing the core in pill form comprising a bioadhesive polymer and at least an active principle formed with a concave side and an opposite flat side.
b) applying the impermeable polymer coating with a spray gun through a rotary cylinder only in the concave face keeping substrate temperature; and
c) leaving the pill flat face without impermeable coating so that bioadhesion and unidirectional controlled release is here allowed towards the absorption mucous membrane.
